(19) United States
(12) Patent Application Publication
(10) Pub. No.: US 2008/0091121 A1
(43) Pub. Date: Apr. 17, 2008

(54) SYSTEM, METHOD AND APPARATUS FOR DETECTING A FORCE APPLIED TO A FINGER

(76) Inventors: Yu Sun, Salt Lake City, UT (US); John Hollerbach, Salt Lake City, UT (US); Stephen Mascaro, Salt Lake City, UT (US)

Correspondence Address: FOLEY & LARDNER LLP, 150 EAST GILMAN STREET, P.O. BOX 1497, MADISON, WI, 53701-1497, US

(21) Appl. No.: 11/688,665

(22) Filed: Mar. 20, 2007

Related U.S. Application Data
(60) Provisional application No. 60/787,996, filed on Mar. 31, 2006.

(51) Int. Cl. A61B 5/00

(52) U.S. Cl. 600/587

(57) ABSTRACT

A system for detecting an amount of force applied to a finger where the back upper portion of the finger is illuminated by light. The system includes intrasubject and intersubject registration. Another embodiment relates to using linear discriminant analysis to generate a fingertip force model and obtain a force measurement. In other embodiments, the system is used to mimic the input of a mouse, a track/touch pad, a keyboard device, or control a robot. In other embodiments, the system is used to assist in physical therapy, medical diagnosis, medical studies, or contact measurement.

### CROSS REFERENCE TO RELATED APPLICATION

The present application claims priority to U.S. Provisional Patent Application No. 60/787,996, filed on Mar. 31, 2006, and titled “A DEVICE AND METHOD OF DETECTING A FORCE APPLIED TO A FINGER,” the contents of which are incorporated herein by reference in their entirety.

### STATEMENT OF GOVERNMENT RIGHTS

This invention was made with United States government support awarded by the following agency: NIH under grant number 1R21 EB004600-01A2. The United States government has certain rights in this invention.

### FIELD

The disclosure is generally directed towards detecting a force applied to a finger and more particularly, to a system, method, and apparatus for detecting a force applied to a finger by observing the coloration of the fingernail and the skin surrounding the fingernail.

### BACKGROUND

In studies relating to human grasping, instrumented objects are typically created that incorporate miniature six-axis force/torque sensors at predefined grasp points. The grasp points of instrumented objects have to be predefined. Force sensors are typically positioned at predefined grasp points. The instrumentation of objects can often be time consuming and expensive.

Studies have shown that coloration changes in the fingernail due to fingertip pressure can serve to transduce fingertip force. Pressure at the finger pad affects blood flow at the fingernail, which causes a non-uniform pattern of color change in the fingernail area. By measuring the intensity of the changes at several points of the fingernail, the fingertip force can be deduced after a calibration procedure.

The relationship between coloration changes in a particular fingernail and a force applied to that finger varies from subject to subject. Furthermore, coloration changes in different fingernails responsive to a force applied to the different fingers for the same subject can vary. Therefore, a calibration procedure is typically required for every fingernail.

In one conventional finger force detection device, an array of LED's for illumination of the fingernail, and an array of photodetectors for detecting light reflected off the surface of the fingernail, are embedded in a custom-fabricated artificial nail made of epoxy. The artificial nail also contains electronics for signal conditioning and a flexible Kapton printed circuit board. The artificial nail attaches to the back of the fingernail with an adhesive, and wires are routed out for interface with a computer. The conventional finger force detection device generated sensor responses that were linear up to 1 N normal force and beyond 1 N there was a nonlinear leveling off. With a linear model, the conventional finger force detection device predicted normal force to within 1 N accuracy in the range of 2 N and predicted shear force to within 0.5 N accuracy in the range of 3 N.

The conventional finger force detection device requires the fabrication of sensors custom fitted to each fingernail and provides relatively sparse sampling of the fingernail. The relatively sparse sampling limits the detection accuracy of coloration changes. The conventional finger force detection device focuses on coloration changes to the fingernail only. Coloration change detection is limited by the fixed placement of the array photodetectors with respect to the fingernail. Coloration changes in the skin surrounding the fingernail have also been found to transduce finger force. Data pertaining to coloration changes in the skin surrounding the fingernail is neither collected nor used to transduce finger force in the conventional finger force detection device.

Besides normal and shear forces, other factors that influence fingernail coloration include shear torque, the contact orientation, the curvature of the contact, and the DIP joint angle. A normal force is labeled as f₂, shear forces are fₓ and f_{y}, shear torque is T₂, fingertip orientation φ^{x} (pitch) and φ^{y} (roll), and finger joint angles J₁, J₂, and J₃ All of the listed factors combine to affect the coloration pattern of the fingernail. The relatively sparse sampling of the fingernail image by conventional systems does not permit the separation of the influences of each of these individual factors in coloration changes. Fingernail coloration also tends to saturate at lower force levels when compared to saturation levels pertaining to that of the skin surrounding the fingernail.

Thus, a system, method, and apparatus for detecting finger force that overcomes one or more of the challenges and/or obstacles described above is needed.

### SUMMARY

An exemplary embodiment relates to a device for detecting an amount of force applied to a finger where the back upper portion of the finger is illuminated by light. The back upper portion of the finger generally consists of a fingernail and the skin surrounding the fingernail. The device includes a photodetector communicatively coupled to a processor. The photodetector is operable to detect a first amount of light reflected back from a back upper portion of a finger and generating a light signal representative of the detected first amount of light. The processor is operable to determine a first amount of force applied to the finger based on the received light signal.

Another embodiment relates to a method of detecting an amount of force applied to a finger where a back upper portion of the finger is illuminated by light. The back upper portion of the finger generally consists of a fingernail and the skin surrounding the fingernail. The method includes detecting a first amount of light reflected back from a back upper portion of a finger at a photodetector and determining a first amount of force applied to the finger based on the detected first amount of light.

Another embodiment relates to a method of registering images both for a single finger (intrasubject) and amongst different fingers and many users (intersubject). Intrasubject registration includes a reference operation, a new image operation, an identification operation, a correlation operation, and a mapping operation. Intersubject registration includes an edge detection operation, a smoothing operation, a segmenting operation, and a mapping operation which produce a registration result.

Another embodiment relates to a method of using linear discriminant analysis to generate a fingertip force model and obtain a force measurement. The embodiment includes a data collection operation, a principle component analysis (PCA) operation, an (LDA) operation, a modeling operation, and a measurement operation.

Another embodiment relates to a method of using a finger force detection input device to interface with a graphical user interface displayed on a display screen. A fingernail and the skin surrounding the fingernail are illuminated. A first amount of light reflected back from the fingernail and the skin surrounding the fingernail is detected at a photodetector. A first finger location of the fingernail and the skin surrounding the fingernail is determined based on the first amount of reflected light. A first cursor location is associated with the first finger location. A second amount of light reflected back from the fingernail and the skin surrounding the fingernail is detected at the photodetector. A second finger location of the fingernail and the skin surrounding the fingernail is determined based on the second amount of reflected light. A first relationship between the first finger location and the second finger location is derived. A second cursor position associated with the second finger location is derived based on the derived relationship.

The applied finger force detection system has applications in the area of human-computer interface. The finger force detection system can be used as an input device for a computer. In one embodiment, the finger force detection system is used to control a cursor of a graphical user interface, mimicking a mouse, a joystick, a stylus, or a puck. In another embodiment, the finger force detection system is used to input text, mimicking a keyboard device. In another embodiment, the finger force detection device is used to control a robot. In other embodiments, the applied finger force detection system is used to assist in physical therapy, medical diagnosis, or medical studies.

Other principal features and advantages of the invention will become apparent to those skilled in the art upon review of the following drawings, the detailed description, and the appended claims.

### BRIEF DESCRIPTION OF THE DRAWINGS

FIG. 1 illustrates a calibration stage in accordance with an exemplary embodiment.

FIG. 2 illustrates an arm support and lighting apparatus in accordance with an exemplary embodiment.

FIG. 3 illustrates a voice coil motor in accordance with an exemplary embodiment.

FIG. 4 illustrates a visual display for guiding subjects through the calibration procedure in accordance with an exemplary embodiment.

FIG. 5A illustrates fiducial marks added to a fingernail in accordance with an exemplary embodiment.

FIG. 5B illustrates a 3D data cloud in accordance with an exemplary embodiment.

FIG. 5C illustrates a registration result in accordance with an exemplary embodiment.

FIG. 6 illustrates a graphical depiction of mapping a 2D image to a 3D model in accordance with an exemplary embodiment.

FIG. 7 illustrates a coloration response of one typical point in a fingernail to a normal force on the finger pad in accordance with an exemplary embodiment.

FIG. 8 illustrates a gradient of a fitted curve in accordance with an exemplary embodiment.

FIG. 9 illustrates a start point map and a saturation point map for one example subject in accordance with an exemplary embodiment.

FIG. 10 illustrates exemplary areas of a finger tip that respond well to the application of different forces in accordance with an exemplary embodiment.

FIG. 11 illustrates individual force predictions for shear forces and a normal force for a first subject in accordance with an exemplary embodiment.

FIG. 11A illustrates a root mean square (RMS) error of prediction results for a first subject and five other subjects in accordance with an exemplary embodiment.

FIG. 12 illustrates experimental force results for one subject in accordance with an exemplary embodiment.

FIG. 13 illustrates histograms of the time constants for forces applied to one particular subject in accordance with an exemplary embodiment.

FIG. 14 illustrates a force prediction result without time compensation in accordance with an exemplary embodiment.

FIG. 15 illustrates a force prediction result with time compensation in accordance with an exemplary embodiment.

FIG. 16 illustrates a time history of a photoplethysmograph in accordance with an exemplary embodiment.

FIG. 17 illustrates a normalized cross-correlation between a photoplethysmograph and a coloration of image pixels for a normal force level of 1N in accordance with an exemplary embodiment.

FIG. 18 illustrates a normalized cross-correlation between a photoplethysmograph and a coloration of image pixels for a normal force level of 6N in accordance with an exemplary embodiment.

FIG. 19 illustrates a camera used in a PUMA 560 simulation in accordance with an exemplary embodiment.

FIG. 20 illustrates a graphical user interface including a virtual control panel displayed on a screen in accordance with an exemplary embodiment.

FIG. 21 depicts a system for measuring force applied by a finger in accordance with an exemplary embodiment.

FIG. 22 depicts a system flowchart describing intrasubject registration in accordance with an exemplary embodiment.

FIG. 23 depicts a system flowchart with pictures describing intersubject registration in accordance with an exemplary embodiment.

FIG. 23A illustrates six raw images and six registration results in accordance with an exemplary embodiment.

FIG. 24 depicts a system flowchart describing a linear discriminant analysis of fingernail images in accordance with an exemplary embodiment.

FIG. 24A illustrates five extracted linear feature vectors in accordance with an exemplary embodiment.

FIG. 24B illustrates a plot of training data projected in a plane spanned by two feature vectors in accordance with an exemplary embodiment.

FIG. 25 depicts a mouse application in accordance with an exemplary embodiment.

FIG. 26 depicts a keyboard application in accordance with an exemplary embodiment.

### DETAILED DESCRIPTION

An apparatus, a system for and method of detecting a force applied by a finger are described. In the following description, for purposes of explanation, numerous specific details are set forth to provide a thorough understanding of exemplary embodiments of the invention. It will be evident, however, to one skilled in the art that the invention may be practiced without these specific details. In other instances, well-known structures and devices are shown in block diagram form to facilitate description of the exemplary embodiments.

An external camera system is used to measure forces applied to a fingertip by imaging the back upper portion of a finger. The back upper portion of a finger generally includes the fingernail and the skin surrounding the fingernail. Calibration results with a force sensor show that the specific measurement ranges depend on the specific regions of the fingernail and the surrounding skin studied for coloration changes. There are dynamic features of the coloration response of different parts of the fingernail and surrounding skin that respond to different force levels. A model predicts the fingertip force associated with given coloration changes. Results for both normal and shear force measurements can also be determined.

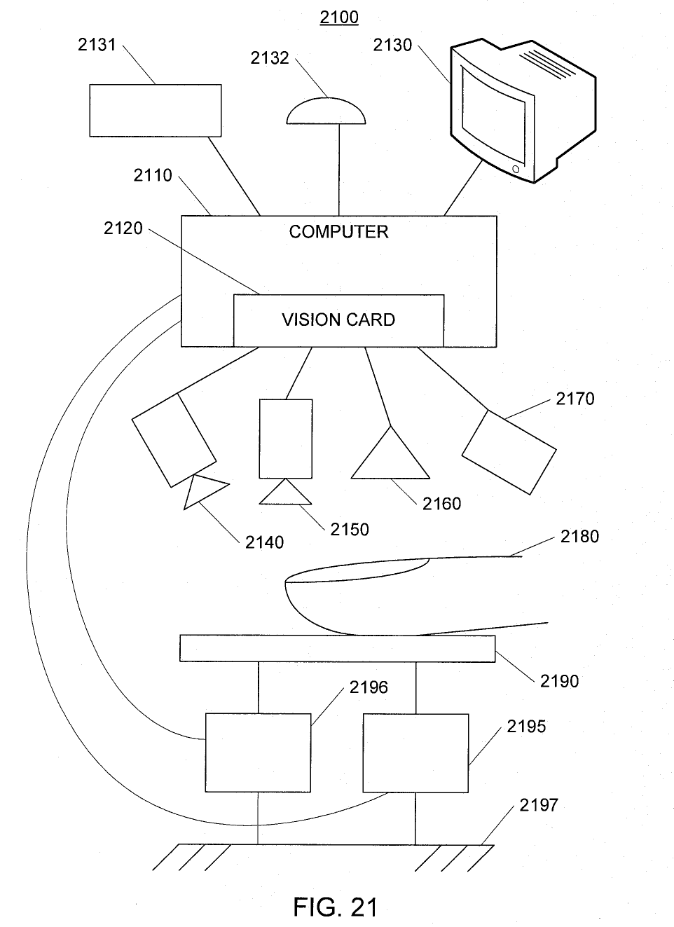

Referring to FIG. 21, a system for measuring force applied by a finger 2100 is shown. A computer 2110 controls the system for measuring force applied by a finger 2100. The computer 2110 includes a vision card 2120 that collects images produced by a video camera 2140 and a stereo video camera 2150. The video camera 2140 is a 1024×768 resolution, color charge coupled device (CCD) camera; however, various resolutions or black and white cameras can be used. Various lenses, filters, shutters, apertures, and polarizers can be mounted on video camera 2140. The lens can be focused manually or with an auto-focusing mechanism. The vision card 2120 and computer 2110 control the intensity and on/off state of a light source 2160 and a grid projector 2170. The video camera 2140, the stereo video camera 2150, the light source 2160, and the grid projector 2170 are all aimed at a stage 2190. The stage 2190 provides a user with a place to position a finger 2180 for examination. A force sensor 2196 and a voice coil motor 2195 are connected between the stage 2190 and a fixed surface 2197. A monitor 2130, a keyboard 2131, and a mouse 2132 allow an operator and the user to interact with the system for measuring force applied by a finger 2100.

The light source 2160 is a light emitting diode (LED) lamp with dome diffuser. Various lighting schemes as known in the art can be used to illuminate the stage 2190 such as LED grids, infrared (IR) lamps, ultraviolet (UV) lamps, diffusers, filters, or lighting elements embedded in the stage 2190 itself. Additionally, the lighting fixtures can be placed at different positions around the stage in order to enhance the illumination of various attributes. Ambient light can also be used to illuminate the stage 2190. The grid projector 2170 is a laser grid projector, but laser line projectors may also be used. The voice coil motor 2195 can be any force application device; however, 6-axis force applicators are preferred where greater precision is desired.

Advantageously, using an external camera system provides relatively fuller imaging compared to prior art devices because the entire fingernail and the skin surrounding the fingernail are imaged, as opposed to just imaging a few sample points of the fingernail. Using an external camera also provides relatively higher resolution images of the fingernail and surrounding skin compared to prior art devices. Furthermore, using an external camera system does not encumber a subject and eliminates the need for sensor fabrication and individual custom fittings. The existence of low-cost cameras and of image processing methods readily performed on personal computers (PCs) makes the instrumentation costs of such an approach relatively low.

The disclosed system and technique using an external camera provides richer data regarding the coloration changes in the fingernail and the skin surrounding the fingernail in response to applied finger forces. Algorithms can efficiently use the richer data supplied by the external camera to make more accurately determinations of applied finger forces.

A fingertip pressing against a 6-axis force sensor was imaged by a camera system in a controlled lighting environment to explore the fundamental effect of fingertip force versus fingernail and surrounding skin coloration. There are both static and dynamic features of the coloration response of the fingernail and surrounding skin to applied fingertip force. A generalized least squares estimation method is developed to predict the force applied to a finger pad from observed coloration changes to the fingernail and the surrounding skin. The application of normal and shear forces to the finger can also be predicted based on the observed coloration changes.

In an alternative embodiment, the camera system is portable and mountable. For instance, the camera system can be mounted on the hand of the user. In another embodiment, the light system is combined with the camera system. The combined system is portable and mountable on the hand of the user.

In another embodiment, a dome light system consists of a reflective hemispherical dome with LED's lining the lower perimeter of the dome. A hole is provided at the center of the hemisphere for the placement of a camera. In another embodiment, the combined light and camera system is mounted on a stand and can be strategically positioned to capture an image of the finger.

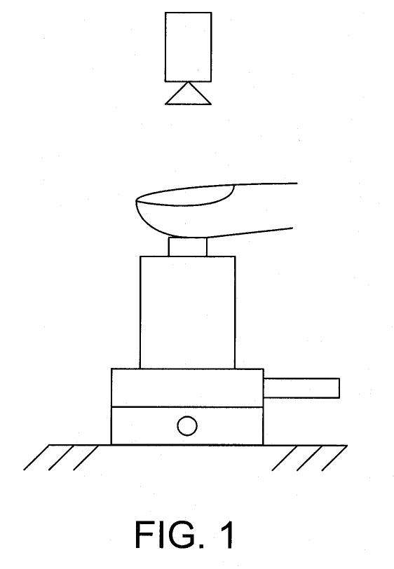

Referring to FIG. 1, a calibration stage including a force sensor, such as a 6-axis JR3 force sensor, mounted on a small manual Cartesian stage, a video camera, such as a Flea CCD video camera (Point Grey Research, Inc.), and a light dome are shown. A rubber-surface flat plane is mounted on the JR3 force sensor to provide a contact surface. The Cartesian table is adjusted to locate the contact plane beneath a subject's fingertip.

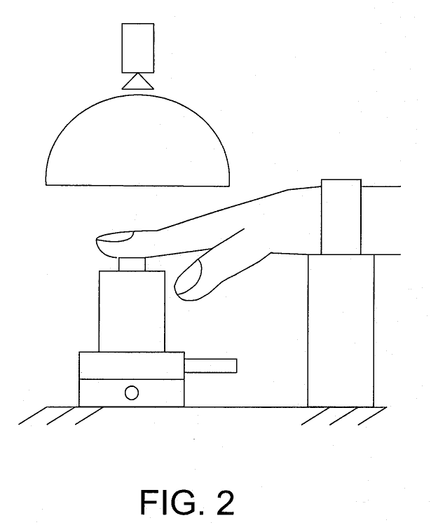

Referring to FIG. 2, an arm support and lighting apparatus are shown. The subject's arm is held in place by a molded plastic arm support and Velcro strips. The plastic arm support has two degrees of freedom (DOF) for position adjustment. A subject sits in a chair adjustable with four DOF for positioning relative to the experimental stage.

The light dome provides a controlled lighting environment so that images taken at different times are comparable. The light dome includes a reflective hemisphere created from molded plastic. A hole provided at the top of the light dome permits visual access by the Flea camera. LEDs are placed along the perimeter of the light dome. The light generated by the LEDs is reflected off the light dome to create uniform lighting on the fingernail surface and the surrounding skin. This arrangement of LEDs minimizes specular reflection.

Images are captured from the Flea camera at 30 fps. When the camera is triggered, the computer also records forces from the JR3 force sensor. The plane of the lens is located approximately 8 cm away from the stage. The field of view on the stage is about 4×3 cm in cross-section. All of the color channels of the camera are used; however, the green channel from the camera's RGB color space typically produces a larger coloration response and better linearity with force than the other color channels.

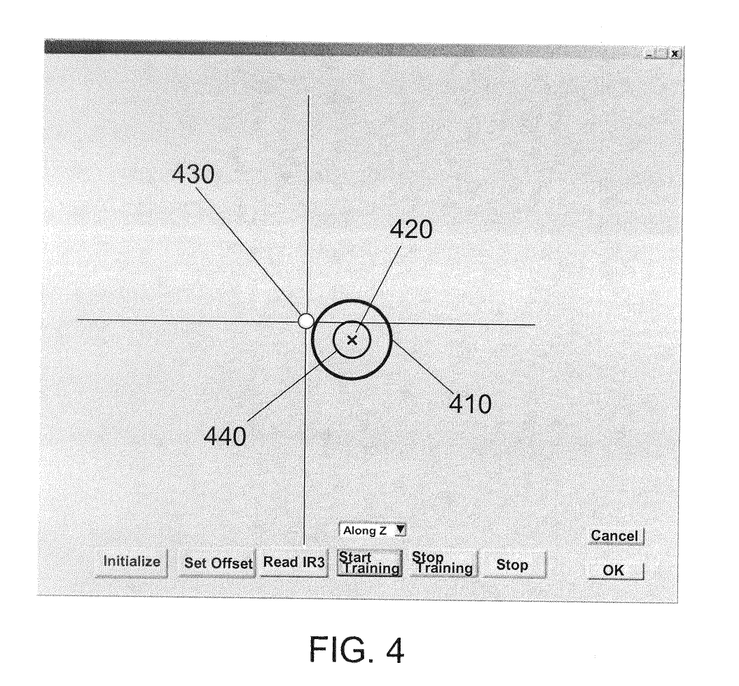

Referring to FIG. 4, a visual display for guiding subjects through the calibration procedure is shown. Two of the three dimensions of force read from the JR3 force sensor are represented by position, while the third dimension is represented by the radius of a circle. Colors may be used in the actual display. In this example, the actual force applied by the user is represented by the circle having the dark outline 410. The cross in the center 420 of the circle having the dark outline represents the actual force applied, as measured by the JR3 force sensor beneath the finger. The x position of the cross represents lateral shear force fₓ, the y position represents longitudinal shear force f_{y}, and the size of the circle represents the normal force f_{z}. The x position of a white-filled circle 430 represents the desired shear force fₓ, and the y position represents desired shear force f_{y}. The circle size of the circle (without a white filling) having a light outline 440 and whose center follows the cross, represents the desired normal force f_{z}. Alternatively, the force reading can be represented textually.

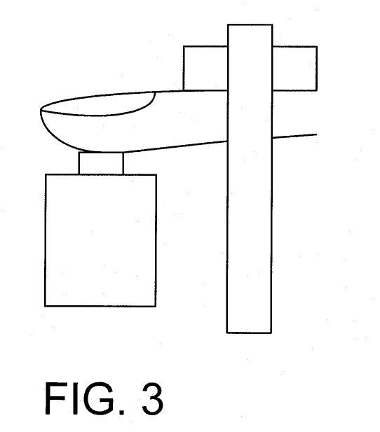

To characterize the dynamic response, a voice coil motor, such as a Bruel & Kjaer 4808 voice coil motor, is used to apply force steps to a fixated finger. An exemplary voice coil motor is shown in FIG. 3. A force sensor, such as a miniature 6-axis AT1 Nano 17 force sensor, records the contact force and is employed in a force controller to regulate contact force.

Image Registration and Surface Modeling

Fingernail locations will vary depending on the different grasp positions and on the relative locations of the camera with respect to the back upper portion of the finger. In one exemplary embodiment, a three dimensional (3D) model of the fingernail surface and surrounding skin is obtained with a stereo camera and laser striping system. Subsequent images from a single camera are registered to the 3D model by adding fiducial markings to the back upper portion of the finger. As a particular fingernail and surrounding skin is imaged, points in the image are correlated to a reference image so that the calibration results can be appropriately applied. The reference image used is a 3D surface model fitted to the back upper portion of the finger because the fingernails and surrounding skin are curved surfaces and the shapes of individual fingernails vary. In one embodiment, a dense triangle mesh model is used. However, alternative models including but not limited to, polygonal meshes, B-spline surfaces, and quadric surfaces, may be used.

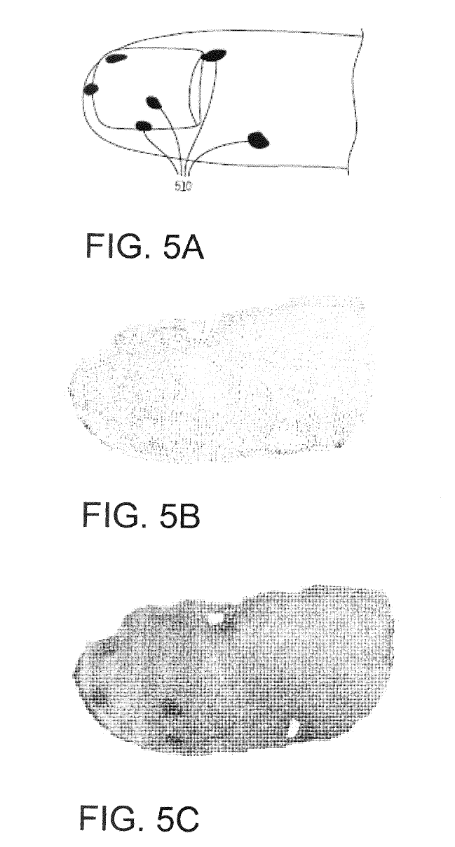

The 3D points that form the vertices of the triangular meshes are obtained with a stereo camera, such as a Bumblebee BB-HICOL-60 (Point Grey Research, Inc.) stereo camera. Since the fingernail is smooth and relatively featureless, it is difficult for the stereo camera system to find corresponding points in the two images. A common computer vision method employed in such situations involves projecting structured light onto the surface, which is easy for stereo vision to match. A laser module, such as a Steminc SMM96355SQR laser module, is used to create a 4-by-4 grid pattern on the surface of a user's fingertip. The stereo images and laser grid are used to create a 3D data cloud as shown in FIG. 5B.

The Bumblebee stereo camera cannot be employed for the coloration measurements because it has a relatively low resolution. However, the data cloud from the Bumblebee stereo camera is adequate for determining a 3D mesh model. Referring to FIG. 5A, to map the high-resolution Flea 2D images to a 3D model, fiducial marks 510 are added to the fingernail and surrounding skin with a black marker. The relative locations of the fiducial marks 510 in the 3D model are obtained using the stereo camera. The fiducial marks 510 are then automatically detected in the 2D image from the Flea camera and used to compute an extrinsic parameter matrix [R t], where R and t are the rotation and the displacement from the 2D image to the coordinates of the 3D model.

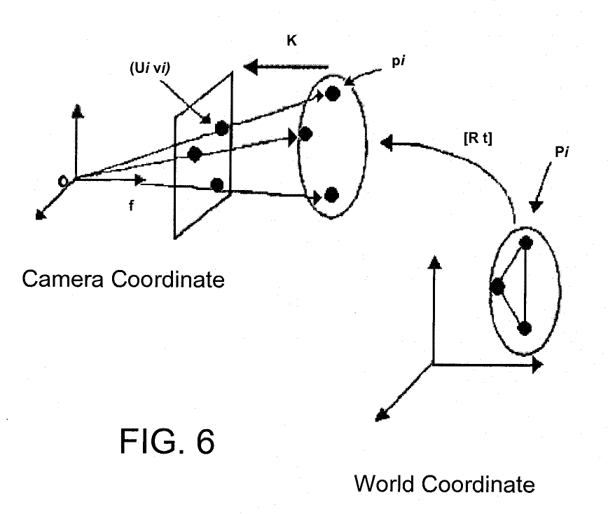

FIG. 6 shows a graphical depiction of mapping a 2D image to a 3D model. The homogeneous coordinates of a point i in the 2D image pi and in the 3D model Pi are:
pⱼ=[uᵢvᵢ1]^{T} Pᵢ=[XYZ1]^{T}

Where the 2D camera coordinates are (uᵢ, vᵢ). Let K be the intrinsic parameter matrix for the camera and define the 3×4 transformation
M=K[Rt]=[m₁m₂m₃]^{T}

The transform relation between the two coordinates is pᵢ=M Pᵢ. Hence,
m₁^{T}Pᵢ−(m₃^{T}Pᵢ) uᵢ=0
m₂^{T}Pi−(m₃^{T}Pᵢ) Vⱼ=0

With six fiducial marks, the parameters in M can be calibrated with linear least squares. FIG. 5C shows a registration result where the 2D image has been fitted to the 3D mesh model.

Coloration Response

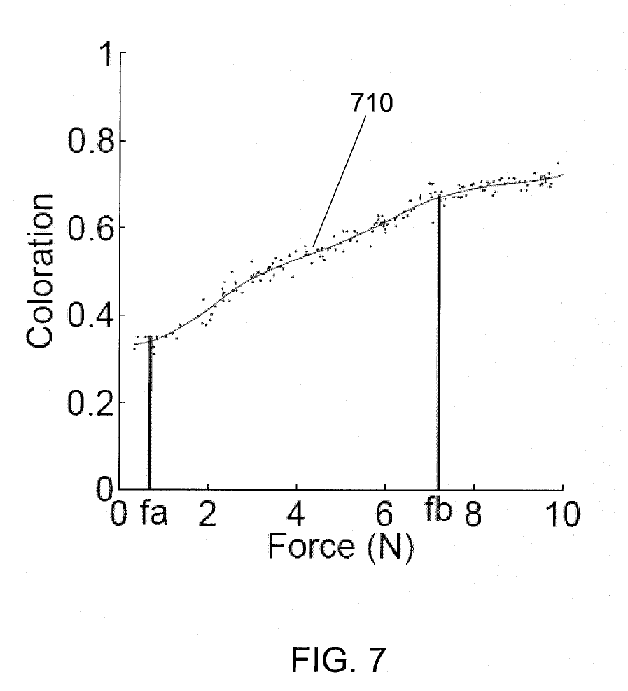

FIG. 7 shows the coloration response hᵢ of one typical point i in the fingernail to a normal force fᵢ on the finger pad. The response curve shows that the coloration starts to change when the force reaches a certain level fₐ and then stops changing at force f_{b} because of saturation. Point i can only transduce the force in the measurement range [fₐ, f_{b}].

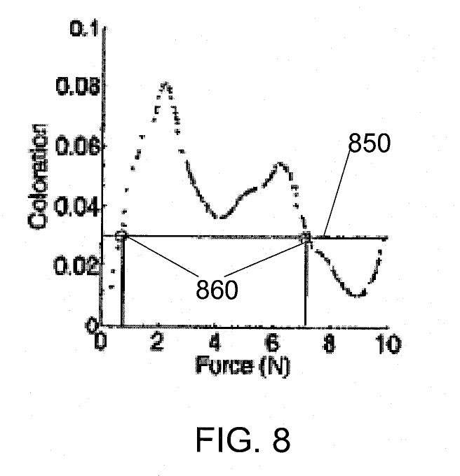

The determination of the response range of a mesh element representing the fingernail and/or its surrounding skin is obtained by thresholding the gradient of its coloration response curve. Locally weighted linear regression is used to fit the data to a response curve. The weighting function is wₖ=exp(−D(fₖ, fᵢ)²/K²_{w}), where i is the index of the query point, and k is the index of points around i. It gives relatively greater weight to the points closer to the query point and relatively less weight to points further away from the query point. This curve fitting emphasizes local information, which can pick up turning points. A typical curve fitting result 710 is shown in FIG. 7. Local gradients on the fitted curve are then calculated using differentials. Referring to FIG. 8, the gradient of the fitted curve is shown. A threshold 850 gₜₕ=0.03 is set. Crossing points 860 where the gradient curve crosses the threshold 850 are found. The measurement range [fₐ, f_{b}] is the segment that starts from a rising crossing point and stops at a falling crossing point. This particular element's measurement range is approximately 1-7 N. Other elements have shown response curves and gradients in a measurement range of approximately 3-6 N.

Different points in the fingernail and surrounding skin have different measurement ranges. Some of them start from a relatively low force, such as, for example, approximately 0 N force, and some of them start from a relatively high force, for example, at approximately 4 N. Some of the points saturate at relatively high applied force levels, for example, at approximately 10 N; while other points saturate at relatively lower force levels, for example, at approximately 3 N. Some points may even have two or more measurement ranges. The largest measurement range of a particular point is defined as the measurement range of that point.

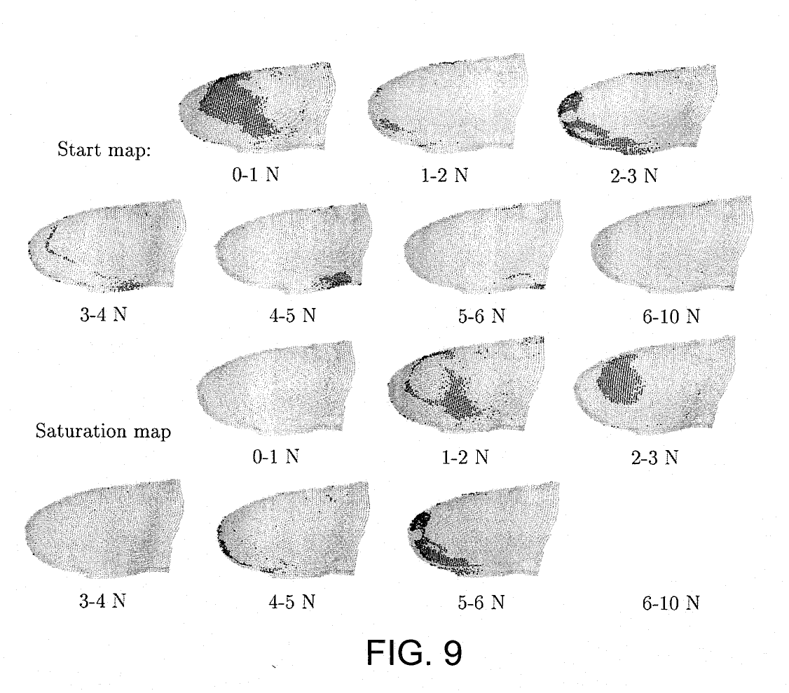

FIG. 9 shows the start point map (top two rows) and the saturation point map (bottom two rows) for one example subject. The dark points in each figure are the regions of the fingernail and surrounding skin with the associated force levels. Most points in the front of the fingernail start to respond at a force level of approximately 2-3 N and saturate at approximately 5-6 N. Most areas in the middle of the fingernail start to respond at approximately 0-1 N. Some of those areas saturate at approximately 1-2 N, while others saturate at approximately 2-3 N. Some areas on the skin surrounding the fingernail start to respond at approximately 3-4 N and some start to respond at approximately 4-5 N. They all saturate at force larger than approximately 6 N.

While this particular subject has specific saturation values, other subjects often have different saturation values than those listed above. For example, some subjects may have a maximum saturation level of approximately 10 N in the skin areas surrounding the fingernail.

There is no single point on the fingernail or the surrounding skin which has a measurement range that covers the entire force range of approximately 0 N to approximately 10 N. Some areas of the back upper portion of the finger have their measurement range at relatively lower level forces, while other areas of the upper finger portion have measurement ranges at relatively higher level forces. By collecting coloration data associated with all of the different areas of the back upper portion of the finger at a given time, the fingernail coloration can be used to transduce forces ranging from approximately 0 N to approximately 10 N for this particular subject.

Linear Response Regions

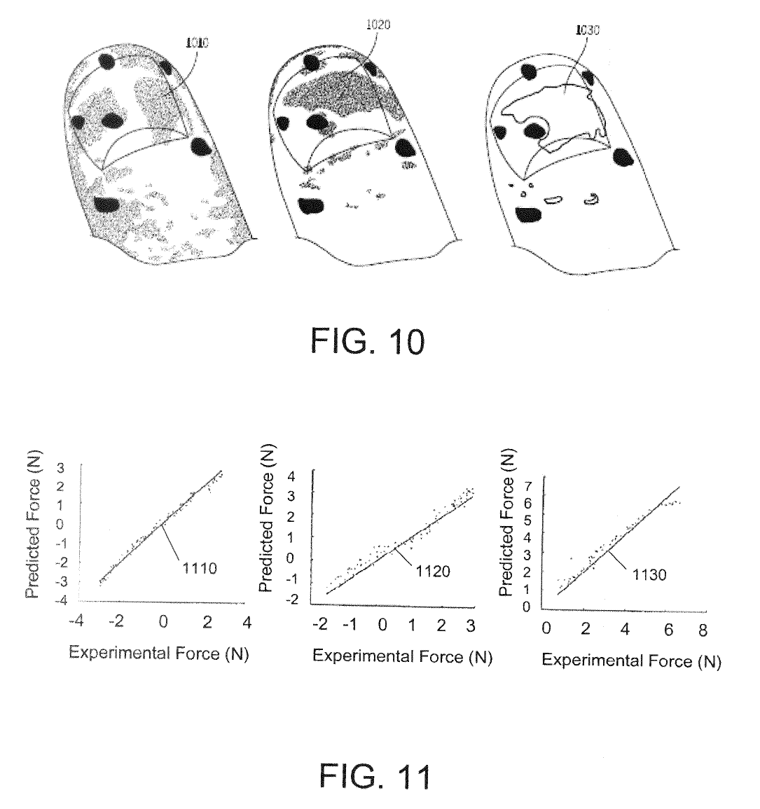

Certain areas of the fingernail and surrounding skin show a relatively superior linear response of coloration to applied fingertip force when compared to others areas of the fingernail and surrounding skin. The location of the good areas to observe coloration depends on the contact conditions. FIG. 10 shows the areas of a finger tip that respond well to the application of different forces. A sideways shear force region 1010 responds to the application of a sideways shear force fₓ. A forward shear force region 1020 responds to the application of a forward shear force f_{y}. A normal force region 1030 responds to the application of a normal force f_{z}. Some areas of the fingernail and surrounding skin respond fairly well to all components of force while other areas of the fingernail and surrounding skin respond particularly well to specific force components. For example, the skin areas surrounding the fingernail are particularly responsive to sideways shear fₓ.

Within the response range of mesh element i, a linear model relating coloration intensity hᵢⱼ to a force component fⱼ for j=1, . . . , n reading pairs are fit using ordinary least squares:
hᵢⱼ=aᵢ₀+aⱼ₁fⱼ, j=1, . . . , n

Where aᵢ₀ and aᵢⱼ are the linear fitting parameters for mesh element i. The quality of fit is determined by thresholding the correlation coefficient rᵢ:

$${r}_{i} = \frac{{\sum }_{j = 1}^{n} ( {f}_{i} - \overline{f} ) ( {h}_{ij} + {\overline{h}}_{i} )}{{( {\sum }_{j = 1}^{n} {( f j - \overline{f} )}^{2} {\sum }_{j = 1}^{n} {( {h}_{ij} + {\overline{h}}_{i} )}^{2} )}^{1 / 2}}$$

Where f̅ and h̅ᵢ are the averages of the force and coloration readings, respectively. A threshold of rᵢ=0.8 is chosen to exclude those mesh elements whose response is not linear enough to be employed for force prediction. In determining the response of cortical neurons to movement parameters a coefficient of determination r²=0.7 is typically used as a threshold to decide which neurons have a good response. A threshold of r²=0.64 is used in this example.

Generalized Least Squares Modeling

Using the good mesh elements with coloration readings hᵢ, a generalized least squares estimator is applied to predict fingertip force vector f=(fₓ, f_{y}, f_{z}) for a single reading. The model is generalized to predict multiple force components:
hᵢ=aᵢ+bᵢ^{T}f+εᵢ
Where aᵢ and bᵢ=(b₁, b₂, b₃) are unknown parameters, and ε, is the residual error. The parameters aᵢ and bᵢ are fitted in their response ranges using ordinary least squares similar to the 1-component force model. Combine all readings hᵢ into a vector h, the parameters aᵢ into a vector a, the parameters bᵢ into the rows of a matrix B, and the errors into a vector ε. Then the stacked equation shown above (hᵢ=aᵢ+bᵢ^{T}f+εᵢ) becomes:
h−a=Bf+ε

Using Q-Q plot, the residual errors of h given a force f satisfy a normal distribution.

$$p ( h \vert f ) = \frac{1}{K} exp - \frac{1}{2} {( h - \overline{h} )}^{T} {Σ}^{- 1} ( h - \overline{h} )$$

Where h̅ is the average of h and K is a constant. The covariance matrix Σ is estimated from the data. The generalized least squares estimate of the force is:
\hat{f}=(B^{T}Σ⁻¹B)⁻¹B^{T}Σ⁻¹(h−a)

The mesh elements are weighted by the uncertainties Σ⁻¹ to produce the best force estimates.

Calibration and Verification

To verify the system, a first set of experiments were carried out to determine how well each force component could be determined in isolation. Six subjects varying in age, size, sex and race participated in the experiments. Subjects used their index fingers to press on the rubber plate mounted on the JR3 force sensor while the camera monitored the coloration changes of the fingernail and surrounding skin of the index finger.

Subjects produced normal force f_{z}, or one of the shear forces fₓ, or f_{y}, under the guidance of visual display feedback. For each of the different directions of a specific force, three sets of data were taken. The first two sets were used for calibration and the third set was used for verification. The estimation equation was simplified to predict only one component of force.

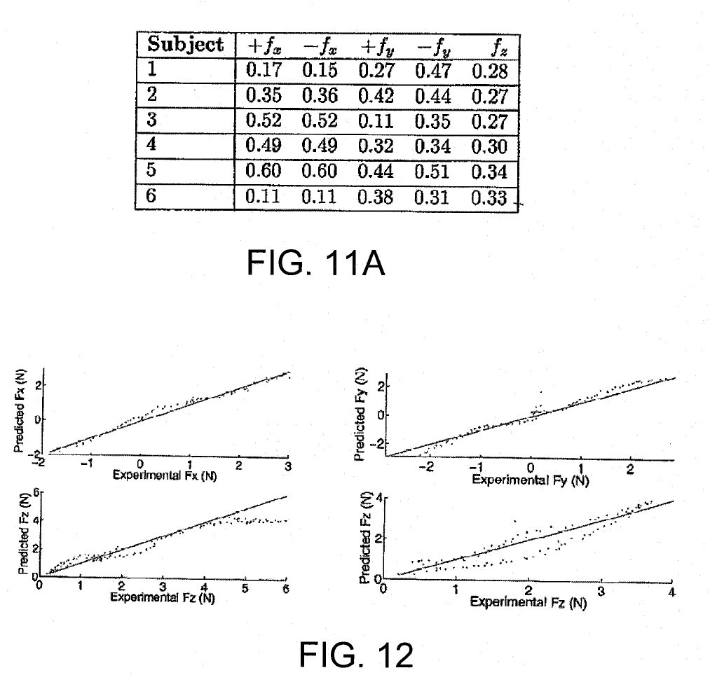

FIG. 11 shows force predictions for shear force (f=fₓ, or f=f_{y},) and normal force (f=f_{z}) for slow force ramps for the first subject. An applied sideways force prediction 1110, an applied forward force prediction 1120, and an applied normal force prediction 1130 all show good linearity. The normal force for this subject saturates above approximately 6 N, which is a typical result. Some subjects have saturation levels as low as approximately 4 N, while others have saturation levels above approximately 8 N. The saturation level limits the magnitude of grasp forces that can be measured by the coloration effect. The shear force levels are less because contact is broken at times between the fingertip and the contact surface. The first subject had a force range of approximately 6 N force. The inaccuracy of prediction ranges from approximately 2.5% (−fₓ) to approximately 7.8% (−f_{y}). The multi-dimensional coefficients of determination were calculated and were found to be above 0.9 and often above 0.95. These numbers illustrate that averaging the responses of the lower-correlation, individual mesh elements has produced the desired effect of increased accuracy. FIG. 11A shows the root mean square (RMS) error of the prediction results for the first subject as well as for the other five subjects.

A second set of experiments were conducted to determine whether a shear force component (either fₓ, or f_{y}) could be predicted simultaneously with normal force f_{z}. Again, the estimation equation was simplified to predict only two force components. The subjects exerted a shear force primarily in either the fₓ direction or the f_{y} direction. The subjects typically also generate some normal force f_{z} as well to maintain frictional contact with the contact surface. A calibration model is developed from one set of data, and then used to predict another data set. FIG. 12 shows the experimental force results for one subject. Responses by the other subjects were similar to that of the one subject. For the x direction, the prediction errors are approximately 0.17 N in fₓ and approximately 0.30 N in f_{z}. For the y direction, the prediction errors are approximately 0.27 N in f_{y} and approximately 0.48 N in f_{z}.

The self-generated shear forces are coupled to the normal forces. This makes it very difficult to estimate the force components separately. A calibration procedure ideally varies the shear/normal force ratio while maintaining frictional contact. Subjects were guided to vary the ratio of shear to normal force using a graphical aid. A motorized calibration stage can be used for calibration to generate a more robust and accurate estimation.

Time Course of the Coloration Effect

The viscoelasticity of the fingertip pulp and circulation dynamics affect the rate of the coloration changes in the fingernail and the surrounding skin in response to changes in applied fingertip force. The mechanical properties of the fingertip were modeled with a viscoelastic model with three time constants. The values of the three time constants were determined to be approximately 0.004 seconds, 0.07 seconds, and 1.4 seconds. Over 75% of the magnitude of the response was due to the first two relatively fast terms, 0.004 seconds and 0.07 seconds, where the time constants are less than 0.1 seconds. From pulsatile pressure variation in the data, the blood flow is already restored by the time that the third term, 1.4 seconds, dominates. The time constant of the response of the blood flow is between approximately 0.1 seconds and approximately 0.4 seconds, depending on which part of the fingernail and surrounding skin is observed.

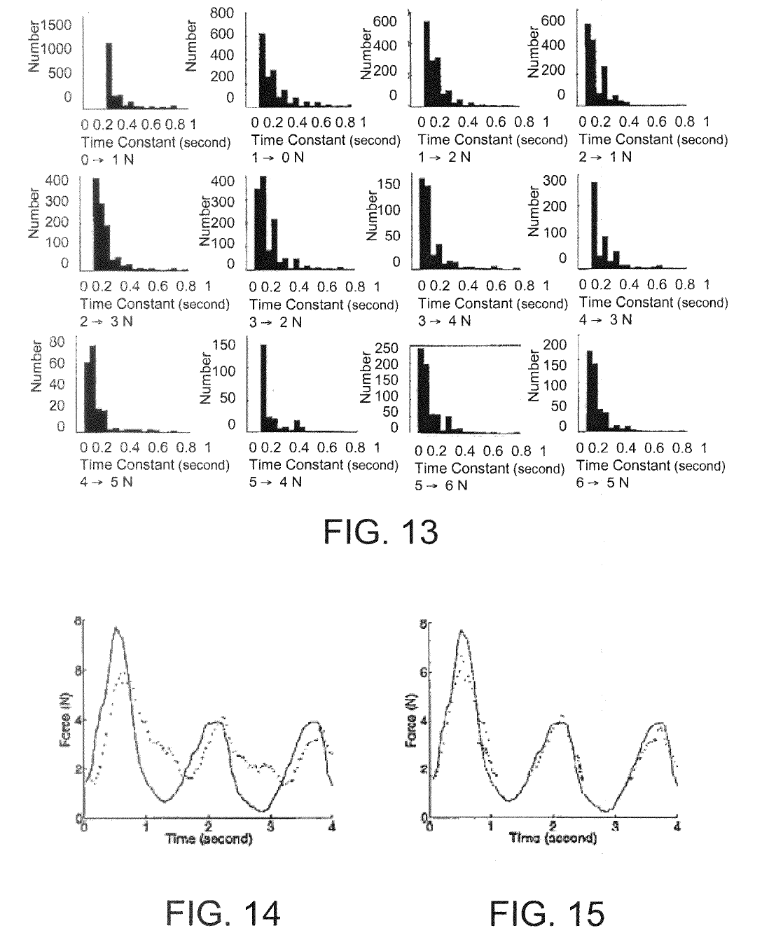

Using the methods described above to identify good mesh elements, it was verified that the coloration effect is reasonably fast. A series of force steps were applied by a linear motor to a fixated finger. For each force step, the first order time constant was calculated for each mesh element. FIG. 13 shows histograms of the time constants for approximately 1 N force steps throughout the force range for both cases of loading and unloading for one particular subject. The time constants tend to cluster around approximately 0.2 seconds, and the loading and unloading responses in the same range are relatively similar. A few mesh elements have responses relatively slower than approximately 0.2 seconds.

Given the time constants of approximately 0.2 seconds or approximately 0.3 seconds, the rate of applied fingertip force change is kept relatively slow to employ the coloration effect. Since the time constants are fairly consistent and are able to be calibrated, for relatively faster force prediction, time compensation can be applied. An experiment was carried out to test how the dynamic features of the fingernail and surrounding skin coloration affect the model and the possibility of time compensation. The training is carried out with slow force and the model is tested on a fast data set. FIG. 14 shows a force prediction result without time compensation. The shapes of the actual versus predicted force are fairly similar, but they are displaced in time. The time compensation result is shown in FIG. 15, and as can be seen there is an improvement when compared to the results without time compensation.

The cardiovascular state of the subject may also affect the measurable coloration of the fingernail and the surrounding skin, particularly ordinary vascular pulsation. Pulsation was measured using transmission photoplethysmography and fingernail and surrounding skin coloration changes were monitored with the Flea camera. The output of the photoplethysmograph and camera were continuously and synchronously recorded for seven to eight seconds while a subject rested his finger on the force sensor, maintaining a constant normal force via display feedback.

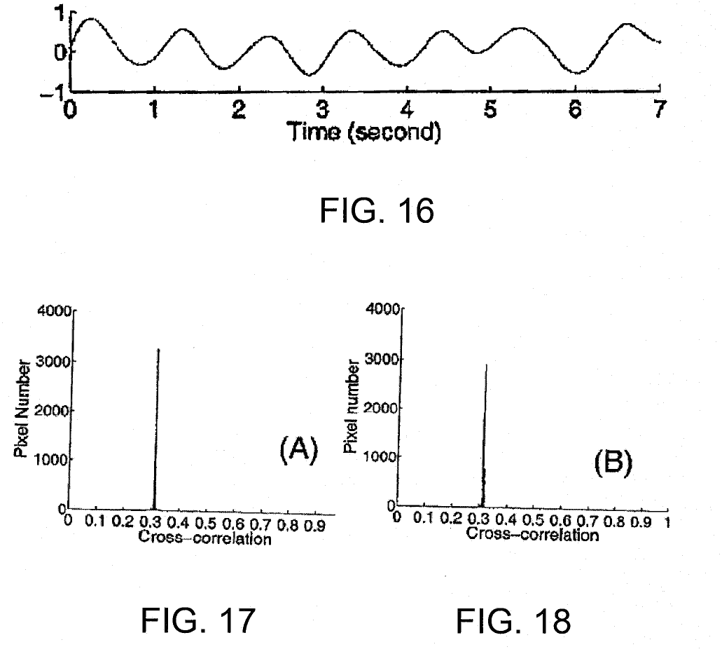

FIG. 16 shows a time history of the photoplethysmograph showing the pulsation. FIG. 17 shows the normalized cross-correlations between the photoplethysmograph and the coloration of the camera pixels for a normal force level of 1 N. FIG. 18 shows the normalized cross-correlations between the photoplethysmograph and the coloration of the camera pixels for a normal force level of 6N. In both cases, the average cross-correlations of all the pixels with the plethysmograph is approximately 0.3, and no pixel correlates more than approximately 0.5. Thus there is no evidence that vascular pulsation affects the coloration changes in the fingernail or surrounding skin in any way that is visible to the camera system.

The use of an external camera system shows a rather complex picture of coloration change with variations in applied fingertip force. Depending on the region of the fingernail and surrounding skin under observation, the usable force range varies. An example of data from a subject shows that the middle region of the fingernail typically has a relatively low force range (approximately 0 N to approximately 2 N), the front region typically has an intermediate force range (approximately 2 to approximately 6 N), and the surrounding skin has a relatively high force range (approximately 3 to greater than approximately 6 N). The saturation point varies from subject to subject. Sometimes the saturation point is less than approximately 6 N, sometimes more. To predict the applied fingertip force response over the entire range from approximately 0 N to saturation, readings from all fingernail and skin regions are combined.

The generalized least square model provides relatively good accuracy for slow force prediction. For all six subjects, the RMS errors are all below approximately 10% and most around approximately 5% of the measuring ranges. Particularly for normal force prediction in isolation, the RMS errors are all around approximately 0.3 N for a measuring range of approximately 6 N to approximately 8 N. The usable force range from the imaging system corresponds well to typical applied fingertip forces during contact. Forces between approximately 0 N to approximately 2 N are typically the most relevant for grasping and typing. A human is typically capable of controlling a constant finger force in the range of approximately 2 N to approximately 6 N with an average error of 6% with visual feedback and natural haptic sense. The force that a human subject can typically comfortably apply for an extended period of time is approximately 3 N.

A greater number of sample points of the fingernail and the skin surrounding the fingernail for coloration observation coupled with the selection of good response regions of the fingernail and skin surrounding the fingernail produce relatively higher force prediction accuracies when compared to those achieved with prior art finger force sensing devices. The generalized least square estimator also yields greater accuracies than the basic least squares estimator. Generalized least squares is only one method for accurate finger force prediction. Bayesian estimation, as well as other estimation procedures, can also be used.

The time course of the coloration affects the prediction accuracy. The dynamic features described above show that for the same measuring point, the time constants are different for different force levels and directions (loading and unloading). The typical time constant is around approximately 0.2 seconds.

The green color channel is often used for coloration observation, since its response range and linearity is relatively better than that of the blue and the red channels. However it should be noted that the use of alternative channels in other color spaces may be used for measuring range and dynamic response features without departing from the spirit of the invention. For example, the hue saturation intensity (HIS) color space may be used.

The disclosed invention can also be used to detect pressure distribution on the fingerpad. For example, in pressure distribution detection, the device can be used to determine whether the contact with the fingerpad is a point contact, a line contact, or contact with a plane. For a point or line contact, the method can also measure the contact location on the fingerpad. When the contact is a flat surface, the technique can detect roughly where the contact is on the fingerpad, such as on the front/back/left/right/center of the fingerpad.

Finger Image Registration

In another exemplary embodiment, advanced registration techniques can be used to enhance accuracy and eliminate the need for fiducial markings. In order to make comparisons between images, both for a single finger (intrasubject) and amongst different fingers and many users (intersubject), the images must be registered. Intrasubject registration registers the subsequent frames of one finger to a reference frame. Intersubject registration registers images of different fingers to an atlas image to obtain common color patterns for all people.

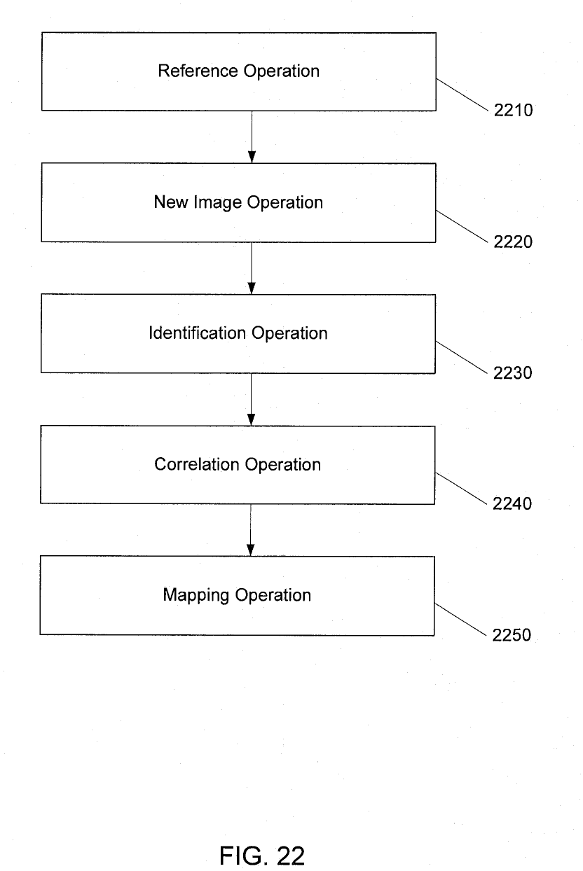

Referring to FIG. 22, a flow chart describing intrasubject registration is shown. In a reference operation 2210, a reference image of the user's finger is captured. As the user moves his finger, a new image is captured in a new image operation 2220. In a feature identification operation 2230, features of the reference image and new image are identified and feature handles (points) are assigned. The Harris feature point detection method is used to automatically detect feature points, although other feature detection methods can be employed. Next, in a correlation operation 2240, the features identified in the reference image are compared to those features in a respective area in the new image. Handles that are maximally correlated with each other are selected as point pairs. The four point pairs with the strongest correlations are selected, but including more point pairs can enhance the registration result. Finally, in a mapping operation 2250, the new image is fit to the reference image using the point pairs. The surface of the fingernail and surrounding skin are assumed to be planar. Hence, the transformation between a point (x₀; y₀) in an additional image and a point (x; y) in the reference image is a homography. RANdom SAmple Consensus (RANSAC) is used to select inliers. The RANSAC algorithm is an algorithm for robust fitting of models in the presence of many data outliers. The inliers are the correspondences. With the correspondences in the new image and the reference image, the 2D homography can be calculated using least squares. Using the homography matrix, the new image is then mapped to the reference image. Operations 2220 through 2250 are repeated for additional new images.

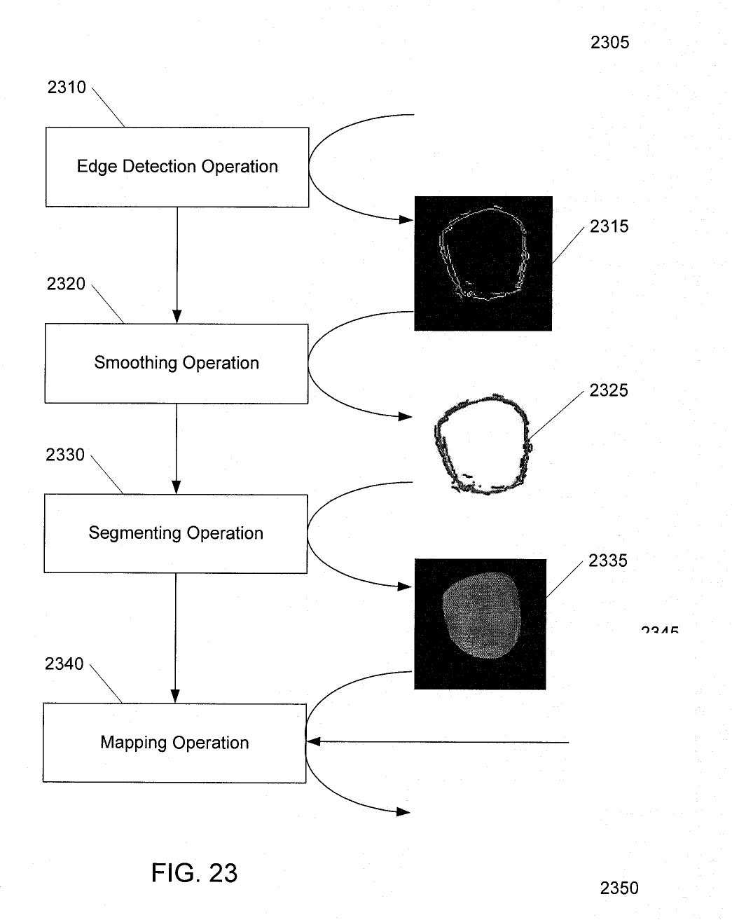

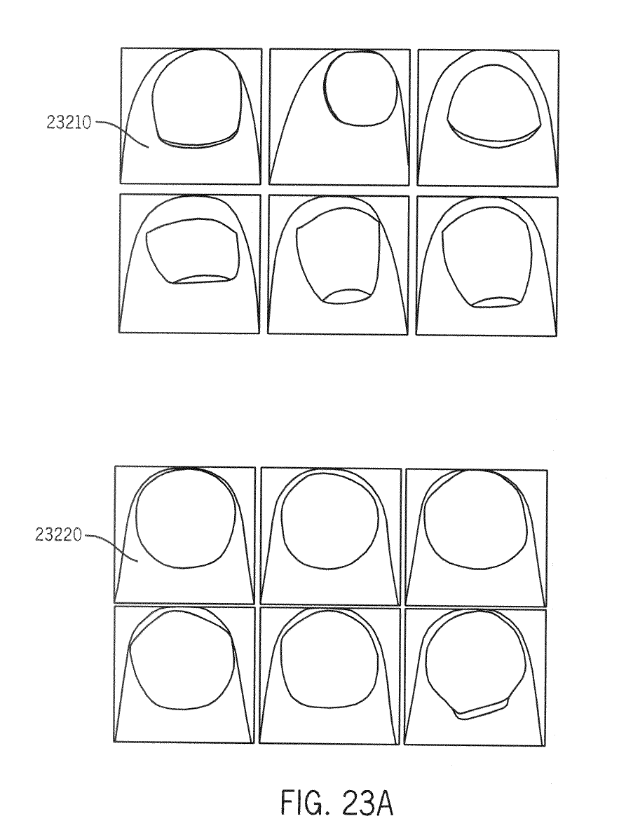

In order to study the color pattern across a population, images of different fingers have to be comparable. Meaningful regions such as the distal, middle and proximal zone of the nail should be consistent for different fingers. Referring to FIG. 23, a flow chart with pictures describing intersubject registration is shown.

In an edge detection operation 2310, an image 2305 is processed using an edge detector in order to find the boundary of the fingernail. In an exemplary embodiment, a canny edge filter is used which produces an edge detection result 2315. In a smoothing operation 2320, the edge detection result 2315 is smoothed into one continuous result. The detected fingernail boundary is typical noisy and can rarely form a smooth curve because of a variety of reasons including broken skin, damaged cuticles, etc. A cubic B-spline is used to fit the edges and archive a closed-loop contour which produces a smoothing result 2325. A variety of different smoothing techniques can be used. Next, in a segmenting operation 2330, the smoothing result 2325 is used to cut out the part of the image that represents the fingernail which produces a segmenting result 2335. Finally, in a mapping operation 2340, the segmenting result 2335 is mapped to an atlas 2345. The atlas 2345 is an anatomical model of a fingertip. The fingernail is modeled as a disk with 70 pixel radius. The surrounding skin region is defined by the circumference of the disk and an isosceles trapezoid. The segmenting result 2335 (which represents the fingernail) and the surrounding skin are transformed to the atlas image, respectively, with boundary-based elastic deformation transformation. The fingernail and surrounding skin regions are modeled as elastic sheets that are warped by an external force field applied to the boundaries. Since elastic warping tends to preserve color pattern shapes and the relative position of the patterns, it is well suited for color pattern comparison across subjects. The boundary of the segmenting result 2335 and boundary of the surrounding skin are homothetically transformed to their respective defined boundaries in the atlas 2345. The boundaries in are first deformed into their corresponding boundaries in the atlas 2345. The mapping of the rest of the segmenting result 2335 is calculated by solving the equations that describe the deformation, which produces a registration result 2350. FIG. 23A shows six before and after images. For example, a raw source image 23210 is converted to a registration result 23220.

Applied Linear Discriminate Analysis

After images of fingertips are collected and registered both intrasubjectly and intersubjectly, force measurements are correlated with the image data. Different directions of force applied on the fingertip change the color patterns in the fingernail and surrounding skin. The different color patterns can be used to classify the finger images into 6 classes corresponding to 6 force directions. Since the color patterns in the images are very high dimensional (where each pixel of an image represents a dimension), a feature extraction method is used to find the features that best describe the color patterns.

Considering that the application of this technique requires real-time processing, a linear feature extraction is preferred although more complex models can be used where computational speed is not important. Moreover, in order to find common color pattern features for all people, the extracted feature should not only maximize the differences between the 6 classes, but also minimize the variation between subjects.

In a preferred embodiment, a linear discriminant analysis (LDA) technique is used to enhance measurement accuracy. The feature extraction problem is the same as the problem of finding projection vectors that maximize the ratio of between-class scatter matrix S_{B} and within-class scatter matrix S_{W}, given by

$$J ( W ) = \frac{\vert {W}^{T} {S}_{B} W \vert}{\vert {W}^{T} {S}_{W} W \vert} \qquad ( 1 )$$

It is the same as

$${J}^{′} ( W ) = \frac{\vert {W}^{T} {S}_{B} W \vert}{\vert {W}^{T} {S}_{T} W \vert} \qquad ( 2 )$$

Where S_{T}=S_{W}+S_{B} is the scatter matrix of the whole data. Finding the vectors to maximize of J′(.) is a generalized eigen-problem. The columns of an optimal W are the C−1 generalized eigenvectors of
S_{B}wᵢ=λᵢS_{T}wᵢ, (3)

Where the C is the number of classes. Here C=6.

Since S_{T} is always singular when the number of training data is smaller than the dimension of the data, a principle component analysis (PCA) is used to reduce the dimension. This is referred to as PCA-LDA. The performance of the PCA-LDA approach heavily depends on the selections of principal components (PCs). A PCA selection scheme based on the correlation between the PCs of S_{T} and the PCs of S_{B} is used.

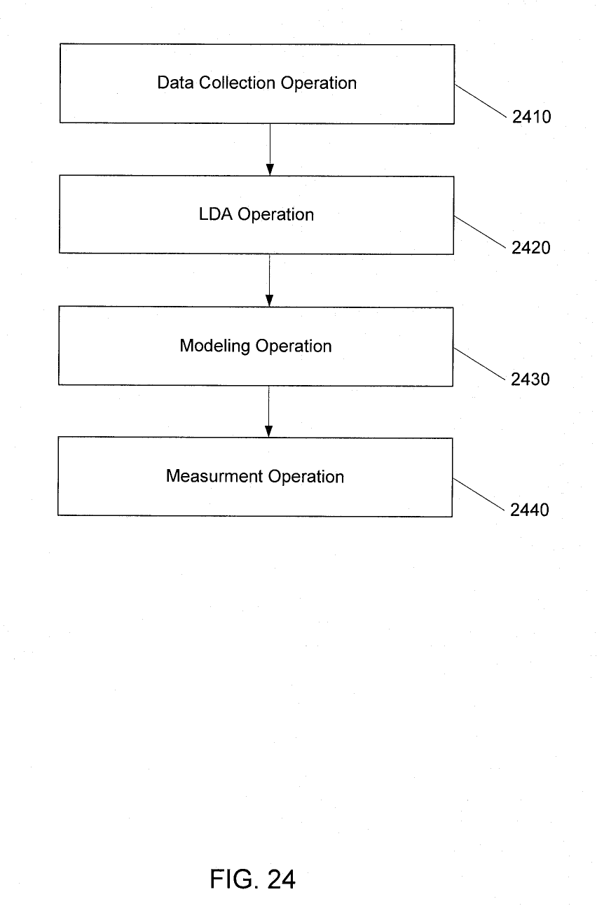

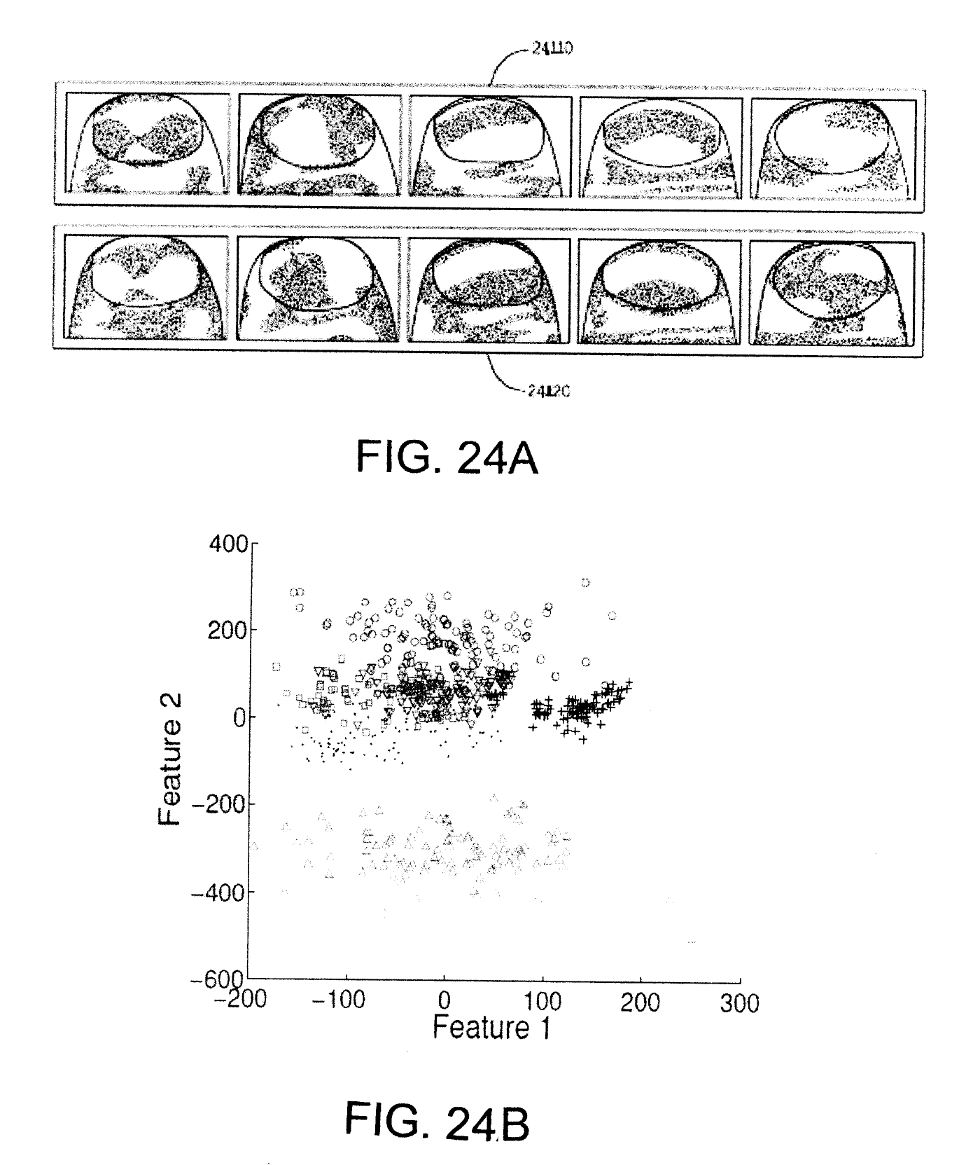

In an exemplary embodiment, referring to FIG. 24, a flow chart describing a linear discriminant analysis of fingernail images is shown. In a data collection operation 2410, a plurality of images is captured. A number of users are prompted to place their finger on the stage and perform a number of force tasks. For instance, the user is prompted to push his finger down with a force of 2N and forward with a force of 3N. A monitor displays the force measurement in order to assist the user. When the task is performed, the system captures an image of the fingertip. For each user, 6 tasks are performed: forward, backward, left right, down, and dead rest. The plurality of images is hereafter referred to as training data.

In an LDA operation 2420, linear discriminant analysis, specifically PCA-LDA, is used to extract features from the training data that reflect the regions most responsive to the force tasks, and that are insensitive to subject differences or environment changes. Using LDA to extract linear features is well-known in the art. The number of the LDA features depends on the number of classes (i.e., the number of classes minus 1). For instance, if there are 6 classes of force directions, there are 5 LDA features. There are many ways to extract LDA features. Alternatively, other classification methods can be used such as LDA, PCA, PCA+LDA, or Support Vector Machine (SVM).

For example, the 2-step LDA procedure called PCA-LDA is used to extract the linear feature vectors. The pixel values of the pixels are the weights from feature vectors. The weights can be positive or negative. An example of five extracted linear feature vectors is shown in FIG. 24A. A top row 24110 shows positive pixel weights and a bottom row 24120 shows the top row's 24110 respective negative weights.

In the present example, the feature space is 5 dimensional. FIG. 24B shows a plot of training data projected in the plane spanned by the first two feature vectors. Notably, the images correlating with the +Fx, −Fx, +Fy and +Fzero force tasks are distinctly grouped. The six clusters represent the force tasks: the lateral shear force directions +Fx (o's) and −Fx (Δ's), the longitudinal shear force directions +Fy (·'s) and −Fy (▾), normal force Fz only (□'s), and no force (+'s). The centroids of the six clusters are then determined.

Referring again to FIG. 24, in a modeling operation 2430, the set of linear feature vectors and the coordinates of the centroids are used to create a fingertip force model. This model is saved for future use.

Finally, in a measurement operation 2440, the model is used to measure the force exerted by a user's fingertip. The user places his fingertip under a camera. The camera captures a new image of the fingertip. The computer then applies the model to the new image. For example, recognition is made in a 5 dimensional space spanned by the Fisher vectors. New images are projected to the Fisher feature space and classified based on the L₂ norm distances to the centroids of the 6 training clusters. A force estimate is generated based on the classifications. The force estimates are then displayed.

In an experiment, 840 new images were captured representing 6 force directions of 7 subjects. The overall accuracy was 92%. For all force directions, the individual accuracies for each subject were over 85%. Four out of seven subjects had recognition accuracy greater than 90%. With respect to the individual force directions, the accuracies of all directions except −Fy were greater than or equal to 94%.

Alternatively, the model can be individualized. In order to increase the model's accuracy in a particular force direction, the centroid for an individual is computed. The new centroid is integrated into the model. Furthermore, the clusters can be analyzed using various methods including a Gaussian Mixture Model. Likewise, non-linear methods of analysis can be employed; the coordinates of the centroid can be expressed as a Euclidean distance.

Human-Computer Interface (HCI) Application

The applied finger force detection system has applications in the area of human-computer interface. The finger force detection system can be used as an input device for a computer. In one embodiment, the finger force detection system is used to control a cursor of a graphical user interface, mimicking a mouse, a track/touch pad, a joystick, a stylus, or a puck. In another embodiment, the finger force detection system is used to mimic a touch screen/touch pad. In another embodiment, the finger force detection system is used to input text, mimicking a keyboard device. In another embodiment, the finger force detection device is used to control a robot. The fingernail and the skin surrounding the fingernail are illuminated using a light source such as the previously described dome light. Alternatively, the fingernail and the skin surrounding the fingernail are illuminated by environmental lighting, such as sunlight or the ambient light in a room.

In the following embodiments, a pre-loaded fingertip force model is assumed. That is, during design and development of the devices, the manufacturer tests a number of subjects and generates a fingertip force model as described above. This fingertip force model is integrated into the device so that a user can take the device out of the box and begin to use the device immediately. Various pre-loaded models can be included that the user selects from. For instance, a user can select a model based on his or her sex and race. Additionally, alternative image registration techniques can be used depending on available processing power.

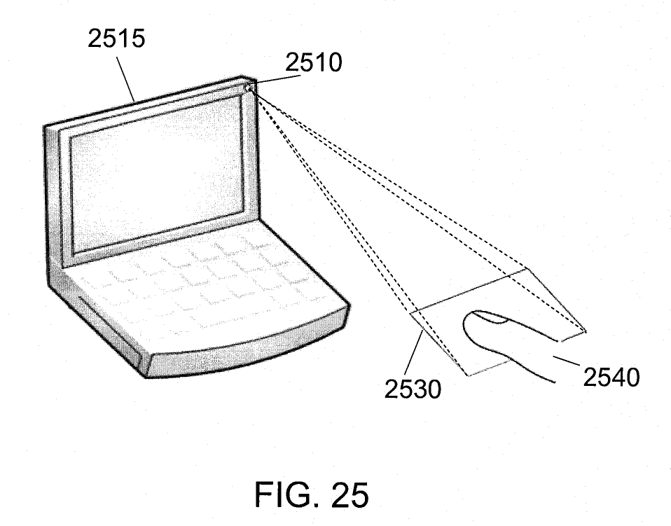

In an exemplary embodiment, the finger force detection system is used like a computer mouse (mouse application). Referring to FIG. 25, a camera 2510 embedded into a laptop computer 2515 is aimed at a particular location that is defined as a control area 2530, which the user knows. The camera 2510 is continuously capturing images of the control area 2530. The user places his finger 2540 in the control area 2530 and lets his fingertip rest. The finger force detection system detects the presence of the finger 2540, and confirms that the object in the control area 2530 is indeed a finger using a object detection algorithm as known in the art.

When a finger object is confirmed, the fingertip image is registered and tested against a model as described above. Hence, the system is constantly determining the amount and direction of the force applied by the fingertip.

The user places a small amount of downward pressure on his fingertip, for instance 1N. The finger force detection system detects that 1N of downward force has been applied. The mouse application is programmed with a number of adjustable thresholds. In this case, the mouse application recognizes that when more than 0.5N of downward force is applied, the user wants to enter a command.

The user now applies a forward sheer force of 1N to his fingertip. The finger force detection system detects that 1 N of forward sheer force has been applied. The mouse application interprets this as moving a mouse “up.” The mouse application instructs the computer to move the cursor towards the top of the screen. When the user returns his finger to rest, the mouse application instructs the computer to stop moving the cursor. Likewise, different amounts of force cause the cursor to move faster or slower. Hence, the mouse application acts similar to a track-point on an IBM (Levono) T-series Thinkpad.

When the user wishes to click on an icon, the user places a greater amount of downward pressure on his fingertip, for instance 2.5N. The mouse application recognizes that when more than 2N of downward force is applied, the user wants to click on something. The mouse application instructs the computer that the user has “clicked.” Additionally, many thresholds can be set and linked to various commands. For example, applying a force of greater than 3.5N means “double-click.”

The mouse application can also be set-up to differentiate between left and right clicks. For example, the user's index finger is currently in a state of 1N downward force. The user lifts his index finger. The mouse application detects no force; additionally, as the finger is lifted towards the camera, the image of the finger gets “larger.” The mouse application can determine how high a finger has been lifted by the width of the finger in the image. The user now moves his index finger to the right and presses downward with a force greater than 2N. The mouse application detects the rightward motion, and detects that a force greater than 2N has been applied. This is interpreted as a “right-click.” Other “clickers” can also be programmed into the mouse application. Likewise, the lifted motion of the finger can be used to control the direction of the cursor and the click instructions.

Although a pre-programmed finger model is assumed, the mouse application can also be trained to respond more accurately to particular users. A training application instructs the user to perform various force tasks as described above. The data from the force tasks are used to generate a new model.

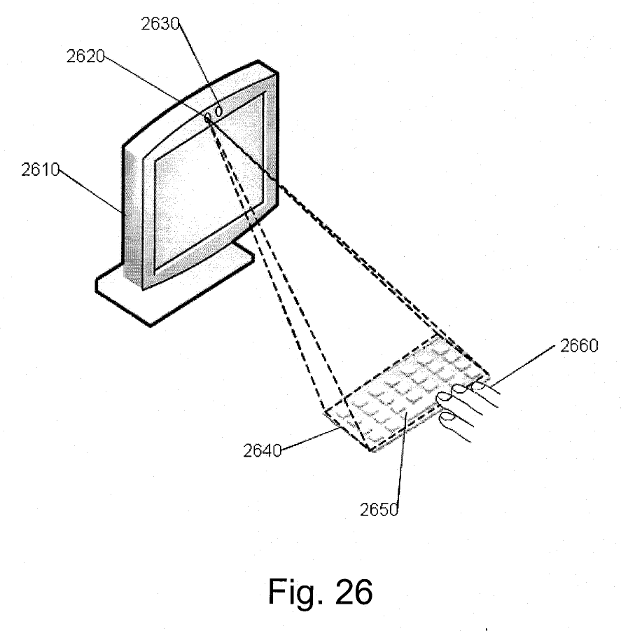

In another exemplary embodiment, the finger force detection system is used like a computer keyboard (keyboard application). Referring to FIG. 26, a camera 2630 embedded into a computer 2610 is aimed at a particular location that is defined as a control area 2640, which the user knows. The control area 2640 may be a picture or an outline of a keyboard 2650. Alternatively, the outline of a keyboard 2650 may be projected onto a surface such as a table or wall by a laser projector 2620.

The camera 2630 is continuously capturing images of the control area 2640. All fingers are imaged simultaneously, but tracked individually. The user places his finger 2660 in the control area 2640. The finger force detection system detects the presence of the finger 2660, and confirms that the object in the control area 2640 is indeed a finger using a modeling algorithm as known in the art. Additionally, the keyboard application determines the location of the finger 2660 relative to the control area 2640.

When a finger object is confirmed, the fingertip image is registered and tested against a fingertip force model as described above. Hence, the system is constantly determining the amount and direction of the force applied by the fingertip as well as the location.

The user places a small amount of downward pressure on his fingertip, for instance 1N, on the “u” area of the control area 2640. The finger force detection system detects that 1N of downward force has been applied. The keyboard application is programmed with a number of adjustable thresholds. In this case, the keyboard application recognizes that when more than 0.5N of downward force is applied, the user wants to enter a command. In this case, the keyboard application interprets this as pressing a key. The keyboard application compares the location of the finger with a map of the control area in order to determine which “key” has been pressed. In this case, the keyboard application determines that a “u” has been pressed and instructs the computer that a “u” has been keyed. Advantageously, as opposed to the prior art, the keyboard application can determine when the user actually intends to press a key.

Additionally, different amounts and direction of force can be programmed to enhance the functionality of the keyboard application. For example, applying a force of greater than 3.5N means “upper-case.” Alternatively, applying a forward sheer force on a key area can be interpreted as holding the shift while pressing the key. For example, applying a forward sheer force on the “5” key would be interpreted as “%.” Other sheer directions, can be programmed for other function keys like the “Control” key.

In another embodiment, the finger force detection system is used like a touch screen or touchpad (touchpad application). The touchpad application has virtual buttons that are considered to be pressed based on fingernail coloration changes. A camera images the fingernail and a user merely presses a blank surface. The user sees a virtual depiction of a real touch panel. An audio signal and/or a blinking icon can signal when a virtual button is considered to be depressed. Touch panels are ubiquitous in everyday environments, including appliances in the home. A simple countertop viewed by a camera becomes a control panel. In the home of the future, with networked appliances, all devices can be controlled from any location chosen by the user, so long as the camera can see the hand and there is an output display of some kind. The display can be a simple analog display such as a LED panel or a liquid crystal display. Many other applications can be envisioned, such as convenient controls for handicapped individuals. The plain arm of a wheelchair, or a table next to a bed, becomes a general-purpose control surface. Moreover, the same surface can be used to represent numerous touchpads.

The mouse application, keyboard application, and touchpad application can be integrated into a device or be attached as an external input device. The mouse application and keyboard application can use the same camera, or photodetector. Exemplary devices include desktop computers, personal digital assistants, calculators, cell phones, music players, or any electronic device that uses human input. Advantageously, the device user does not need to carry a mouse or keyboard when traveling, saving space and weight. Furthermore, any surface can be turned into an input device.

In another embodiment, the finger force detection device is used to control a robot. One example of a graphical user interface including a virtual control panel is displayed on a screen as shown in FIG. 20. The graphical user interface is designed to use the observed relative movements of a finger and coloration changes associated with detected applied finger pressure as inputs to control a device, such as for example, a PUMA 560 simulation.

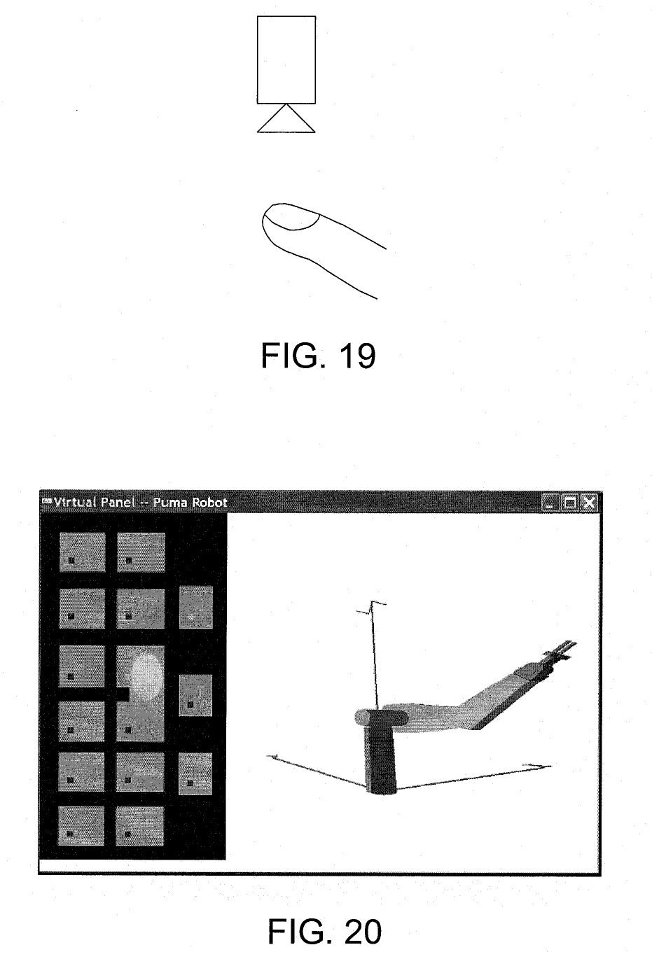

The virtual panel includes a virtual finger to represent the position of a cursor on the display screen. A PUMA 560 simulation and a camera are shown in FIG. 19. The camera tracks the position of the fingertip in the view and tests whether the finger is pressing or not in real time. The location of the fingertip in the view of the camera is converted to the location of the virtual fingertip on the virtual panel.

In one example of a display, if a finger pressing (in other words, the application of force to the fingerpad) is detected via observed coloration in the fingernail and the skin surrounding the fingernail of the user's finger (back upper portion of the user's finger), the color of the virtual fingernail changes to white on the display screen. If the virtual fingertip on the display screen is right on a functional button at the time, the LED on the functional button below the virtual fingertip lights up and a command associated with the user selected function button, such as for example a rotation command, is issued to the PUMA 560 simulation.

The applied finger force detection algorithm monitors the color pattern on the fingernail and surrounding skin and uses the coloration distribution information to classify the input status of the user's finger. Since most people typically have a similar coloration patterns on their fingernails and surrounding skin when force is applied to the finger, the applied finger force detection input device does not require calibration. A demo system was tested on a number of subjects without any pre-knowledge or calibration. All of the subjects were able to use the applied finger force detection interface to control the behavior of the PUMA 560 robot simulation.

The human-computer interface application of the applied finger force detection device typically uses a commercial camera to detect and track the 2D movement and 2D orientation of human finger. The 3D force direction of the applied finger force is estimated by monitoring the coloration of the fingernail and the surrounding skin. A total of 6-DOF inputs are used. As described above, different regions of the fingernail and the skin surrounding the fingernail have different linear responses to different directions of force. The 3D force can be decoupled in the training and estimation thereby minimizing the amount of training that users will require to use some simple settings.

A pattern recognition algorithm can be used to associate different coloration patterns on fingernails and the skin surrounding the fingernail with (i) no application of force to the finger (no pressing with the finger); (ii) the application of force to the finger (pressing with the finger); (3) latitude shear force exerted by the finger; and (4) longitudinal shear force exerted by the finger. The classification of the colorations changes to the fingernail and the skin surrounding the fingernail may be used to provide 3D inputs to a graphical user interface.

In other embodiments, the applied finger force detection system is used to assist in physical therapy, medical diagnosis, medical studies, or contact measurement. For example, in a physical therapy context, a patient is asked to grip a target. The finger force detection system captures an image of the patient's hand at dead rest on the grip. The image is segmented into the individual fingers. Each segment is registered and then processed as described above.

The patient is then asked to apply a gripping force to the target. Again, the finger force detection system captures an image, segments the image, and then calculates the force applied by the individual fingers of the patient. Advantageously, the physical therapist can easily determine which fingers need therapy. Also, multiple targets can be used with one system. The finger force detection allows for targets made of cheap plastic materials which can be easily customized for a particular patient, whereas current force sensors are expensive to build and customize for individual patients.

The finger force detection system can also be used to monitor circulation in fingers or other hand problems. For example, a patient is having circulation problems. In order to track the progress of the patient, the doctor orders a finger force survey. The finger force survey prompts the patient to complete various force tasks. The images are recorded for different force tasks. The doctor uses this survey as a reference.

After therapy, surgery, or drug treatment, the doctor has the patient complete another finger force survey. Using the reference, the doctor can determine the progress of the patient and whether or not his treatment is working. For a given force task, there are two images, the reference image and the progress image. The coloration difference is used to determine the change.

Likewise, the finger force detection system can also be used to monitor the effects of a drug. For instance, a drug that increases circulation is tested. First, the patient is given a finger force survey without the drug. This survey is used as a reference. The drug is administered. At different time periods, the finger force survey is repeated. Hence, the researchers can track the effects of the drug over time.

In another embodiment, the contact location and type can also be estimated using the finger force detection system. The applied linear discriminate analysis is performed using finger pad tasks instead of force tasks (although tasks are normalized with respect to force applied). For example, the user is told to roll his finger to the left. The finger force detection system captures an image. The contact area of the finger pad is also captured either by a camera or a sensor. Likewise, the user performs additional finger pad tasks in other directions. The LDA operation is used to correlate changes in finger coloration to the location and type of the finger pad contact area. Hence, by observing only the fingertip, the contact area can be determined.

The foregoing description of exemplary embodiments have been presented for purposes of illustration and of description. It is not intended to be exhaustive or to limit the invention to the precise form disclosed, and modifications and variations are possible in light of the above teachings or may be acquired from practice of the invention. For instance, the embodiments may be applied to other appendages such as the toe. The functionality described may be implemented in a single executable or application or may be distributed among modules that differ in number and distribution of functionality from those described herein. Additionally, the order of execution of the functions may be changed depending on the embodiment. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed:

1. A device for detecting an amount of force applied to a finger, a back upper portion of the finger being illuminated by light where the back upper portion of the finger consists of a fingernail and skin surrounding the fingernail, the device comprising:
a photodetector operable to detect a first amount of light reflected back from a back upper portion of a finger and generating a light signal representative of the detected first amount of light; and
a processor communicatively coupled to the photodetector and operable to determine a first amount of force applied to the finger based on the received light signal.

2. The device of claim 1, further comprising a light source for illuminating the back upper portion of the finger.

3. The device of claim 2, wherein the light source comprises a light source having a spectral range including visible and infrared wavelengths.

4. The device of claim 1, where the processor determines a first amount of lateral shear force applied to the finger based on the received light signal.

5. The device of claim 1, wherein the processor determines a first amount of longitudinal shear force applied to the finger based on the received light signal.

6. The device of claim 1, wherein the processor determines a first amount of normal force applied to the finger based on the received light signal.

7. The device of claim 1, wherein the processor is operable to generate a first signal when the determined first amount of force exceeds a threshold value and is operable to generate a second signal when the determined first amount of force is below the threshold value.

8. The device of claim 1, further including a second photodetector.

9. The device of claim 1, wherein the processor is further configured with instructions to detect a first amount of light reflected back from a back upper portion of a finger at a photodetector and determine a first amount of force applied to the finger based on the detected first amount of light.

10. A method of using a finger force detection input device to interface with a graphical user interface displayed on a display screen, the method comprising:
illuminating a fingernail and skin surrounding the fingernail;
detecting a first amount of light reflected back from the fingernail and the skin surrounding the fingernail at a photodetector;
determining a first finger location of the fingernail and the skin surrounding the fingernail based on the first amount of reflected light;
associating a first cursor location with the first finger location;
detecting a second amount of light reflected back from the fingernail and the skin surrounding the fingernail at the photodetector;
determining a second finger location of the fingernail and the skin surrounding the fingernail based on the second amount of reflected light;
deriving a first relationship between the first finger location and the second finger location; and
deriving a second cursor position associated with the second finger location based on the derived relationship.

11. The method of claim 10, further comprising:
detecting a third amount of light reflected back from the fingernail and the skin surrounding the fingernail at a photodetector;
determining a first amount of force applied to the finger based on the third amount of reflected light;
generating a first signal when the determined first amount of force exceeds a pre-defined threshold value; and
generating a second signal when the determined first amount of force is below the pre-defined threshold value.

12. The method of claim 10, further comprising displaying a virtual fingertip at the first cursor position on a display screen.

13. A system for creating a finger force model comprising:
an image capturing device configured to capture an image of an entire fingernail and skin surrounding the fingernail;
a force sensor; and
a processor including programmed instructions for creating a finger force model and using the finger force model to obtain force measurements.

14. The system of claim 13, wherein the processor further includes instructions for:
reading the force sensor and creating force data; and
capturing an image from the image capturing device, the captured image being included with image data.

15. The system of claim 14 wherein the response algorithm is an analysis-linear discriminate analysis (PCA-LDA).

16. The system of claim 14 wherein the processor further includes instructions for:
identifying a finger in the image data; and
registering the finger in the image data.

17. The system of claim 14 wherein the finger force model comprises:
plurality of features constituting a feature space; and
plurality of projections, projecting into the feature space.

18. The system of claim 17 wherein at least one of the projections in the plurality of projections is based upon the force data and the image data derived from a single subject.

19. The system of claim 14 wherein the finger force model comprises:
plurality of features constituting a feature space; and
continuous projections which project into the feature space.

20. The system of claim 16 wherein registering the finger in the image data comprises an intrasubject registration.
